(19) United States
(12) Patent Application Publication
(10) Pub. No.: US 2007/0088021 A1
(43) Pub. Date: Apr. 19, 2007

(54) 4-haloisoquinoline derivative and drug containing the same

(76) Inventors: Hiroyoshi Hidaka, Nagoya-shi (JP); Masahiro Tamura, Tokyo (JP); Hiroshi Nakao, Tsuchiura-shi (JP); Hiromichi Sigyo, Tokyo (JP); Hajime Yamada, Tokyo (JP); Takatoshi Ozawa, Tokyo (JP); Hideo Yoshizaki, Sayama-shi (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/518,141

(22) Filed: Sep. 11, 2006

Related U.S. Application Data
(63) Continuation-in-part of application No. PCT/JP06/08566, filed on Apr. 25, 2005.

(30) Foreign Application Priority Data
Apr. 25, 2005 (JP) 2005-126495

(51) Int. Cl. A61K 31/551

(52) U.S. Cl. 514/218

(57) ABSTRACT

The present invention relates to compounds which have a potent Rho-kinase inhibitory action and which are useful as therapeutic agents for treating diseases, such as hypertension, pulmonary hypertension, cerebral vasospasm, angina pectoris, cardiac failure, arteriosclerosis, glaucoma, dysuria, asthma, and erectile failure, and drugs containing the compounds.
The present invention provides a 4-haloisoquinoline derivative represented by formula (1): 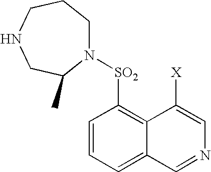 an acid-added salt thereof, or a solvate of any of the foregoing, in which X is halogen, such as fluoro, chloro, bromo and iodo.

### CROSS REFERENCE TO RELATED APPLICATIONS

This application is a continuation-in-part application of PCT/JP2006/308566 filed on Apr. 24, 2006, which is incorporated herein by reference.

### TECHNICAL FIELD

The present invention relates to compounds which have a potent Rho-kinase inhibitory action and which are useful as therapeutic agents for treating diseases, such as hypertension, pulmonary hypertension, cerebral vasospasm, angina pectoris, cardiac failure, arteriosclerosis, glaucoma, dysuria, asthma, and erectile failure, and drugs containing such compounds.

### BACKGROUND OF THE INVENTION

Rho, which is a low-molecular-weight GTP-binding protein, is activated by signals from various cell membrane receptors and converted from inactive Rho-GDP to active Rho-GTP. It has been revealed that the activated Rho in turn activates a Rho kinase downstream of Rho and acts as molecular switches for various cellular phenomena, such as smooth muscle contraction through the actomyosin system, cell motility, cell adhesion, cellular morphological changes, and cell growth. Consequently, it is considered that if Rho kinase is inhibited, responses of various cellular phenomena present downstream of the information transmission pathway through Rho can be suppressed, and thus the inhibition of Rho kinase is effective in treating diseases in which Rho is involved.

For example, when Rho kinase is activated, smooth muscle contracts. When this enzyme is inhibited, smooth muscle relaxes. This is considered to be caused by an action mechanism that increases in Ca ion sensitivity through G-proteins (guanine nucleotide-binding regulatory proteins) are selectively inhibited, and thus Ca ion sensitivity in the cells decreases. It has been reported in papers that Rho kinase acts selectively on the Ca-ion-sensitivity-increasing mechanism, which is one of the smooth muscle contraction mechanisms that do not depend on the intracellular Ca ion concentration (for example, refer to Non-Patent Document 1). Therefore, compounds that inhibit Rho kinase are considered as promising therapeutic agents having a new mechanism that works by decreasing Ca ion sensitivity, for example, therapeutic agents for treating hypertension, etc.

According to recent research, in addition to the blood pressure-decreasing effect realized by inhibiting Rho kinase (for example, refer to Non-Patent Documents 2 and 3), the following has also been reported: cases showing effectiveness in treating pulmonary hypertension (for example, refer to Non-Patent Documents 4 to 6), cases showing effectiveness in treating cerebral vasospasm (for example, refer to Non-Patent Documents 7 and 8), cases showing effectiveness in treating angina pectoris (for example, refer to Non-Patent Documents 9 to 11), cases showing effectiveness in treating glaucoma (for example, refer to Non-Patent Documents 12 to 14), cases showing effectiveness in treating dysuria (for example, refer to Non-Patent Document 15), cases showing effectiveness in treating asthma (for example, refer to Non-Patent Documents 16 to 19), cases showing effectiveness in treating erectile failure (for example, refer to Non-Patent Documents 20 and 21), and the like.

Currently, fasudil hydrochloride (trade name: Eril injection), which is a Rho-kinase inhibitor, is widely used for an indication of “improvement of cerebral vasospasm after subarachnoid hemorrhage surgery and cerebral ischemia associated with the cerebral vasospasm”. The 50% inhibitory concentration (IC50; μM) of this compound for Rho kinase, however, is low being of the order of 1/10. Furthermore, although Rho-kinase inhibitors described in Patent Document 1 are disclosed to be effective for preventing or treating asthma, the 50% inhibitory concentration (IC50; μM) of the compounds disclosed for Rho kinase in this reference is of the order of 1/100 even at the highest level (for example, (S)-(+)-hexahydro-2-methyl-1-(4-methyl-5-isoquinolinesulfonyl)-1H-1,4-diazepine hydrochloride), which is unsatisfactory as an Rho-kinase inhibitor.

Therefore, it has been desired to develop a more potent Rho-kinase inhibitor in which the dose can be decreased when an Rho-kinase inhibitor is actually administered as a therapeutic agent, and the risk of side effects due to the decrease in the dose can be reduced.

Patent Document 1: Japanese Unexamined Patent application Publication No. 11-349482

Non-Patent Document 1: Nature 389(1997): 990

Non-Patent Document 2: J. Cereb. blood Flow 21(2001): 876

Non-Patent Document 3: Hypertension 38(2001): 1307

Non-Patent Document 4: Atheroscierosis Supplements 4(2003): 170

Non-Patent Document 5: Ir. J. Med. Sci. 172(2003): 20

Non-Patent Document 6: Circ. Res. 94(2004): 385

Non-Patent Document 7: Br. J. Pharmacol. 130(2000): 219

Non-Patent Document 8: Stroke 32(2001): 2913

Non-Patent Document 9: Jpn. J. Pharmacol. 87(2001): 34

Non-Patent Document 10: Br. J. Pharmacol. 134(2001): 1724

Non-Patent Document 11: Circulation 105(2002): 1545

Non-Patent Document 12: Invest. Ophthalmol. Visual Sci. 42(2001): 137

Non-Patent Document 13: Arch. Ophthalmol. 119(2001)

Non-Patent Document 14: 1171, Invest. Ophthalmol. Visual Sci. 42(2001): 1029

Non-Patent Document 15: Br. J. Pharmacol. 143(2004): 431

Non-Patent Document 16: Jpn. J. Allergol. 48(1999): 1079

Non-Patent Document 17: Eur. J. Pharmacol. 389(2000): 103

Non-Patent Document 18: Eur. J. Pharmacol. 406(2000): 273

Non-Patent Document 19: Br. J. Pharmacol. 132(2001): 111

Non-Patent Document 20: Int. J. Impot. Res. 13(2001): 67

Non-Patent Document 21: Br. J. Pharmacol. 133(2001): 455

### SUMMARY OF THE INVENTION

It is an object of the present invention to provide compounds having potent Rho-kinase inhibitory action and drugs containing them.

Under these circumstances, the present inventors have conducted intense research and, as a result, have found that the compound represented by formula (1) below has a potent Rho-kinase inhibitory action and is useful as a therapeutic agent for treating diseases, such as hypertension, pulmonary hypertension, cerebral vasospasm, angina pectoris, cardiac failure, arteriosclerosis, glaucoma, dysuria, asthma, and erectile failure. The present invention has thus been completed.

Namely, the present invention provides a compound represented by formula (1): 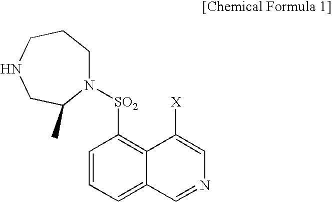
its acid-added salt, or their solvates, in which X is halogen, such as fluoro, chloro, bromo and iodo.

Furthermore, the present invention provides drugs containing, as an active ingredient, the compound represented by formula (1), its acid-added salt, or their solvates.

Furthermore, the present invention provides therapeutic agents for treating a disease caused by the activation of Rho kinase, which contain, as an active ingredient, the compound represented by formula (1), its acid-added salt, or their solvates.

Furthermore, the present invention provides pharmaceutical compositions containing the compound represented by formula (1), its acid-added salt, or their solvates, and a pharmaceutically acceptable carrier.

Furthermore, the present invention provides uses of the compound represented by formula (1), its acid-added salt, or their solvates in manufacture of drugs.

Furthermore, the present invention provides methods for treating a disease caused by the activation of Rho kinase, which include administering the compound represented by formula (1), its acid-added salt, their solvates.

The compound (1) of the present invention, its acid-added salt, or their solvates has a potent Rho-kinase inhibitory action and is useful as a therapeutic agent for treating diseases, such as hypertension, pulmonary hypertension, cerebral vasospasm, angina pectoris, cardiac failure, arteriosclerosis, glaucoma, dysuria, asthma, and erectile failure.

### DETAILED DESCRIPTION

As the acid-added salt of the compound (1) of the present invention, any pharmaceutically acceptable salt can be used without limitations. Its examples include acid-added salts of mineral acids, such as hydrochlorides, hydrobromides, hydroiodides, sulfates, and phosphates; and acid-added salts of organic acids, such as benzoates, methanesulfonates, ethanesulfonates, benzenesulfonates, p-toluenesulfonates, oxalates, maleates, fumarates, tartrates, citrates, and acetates.

Furthermore, the compound (1) of the present invention can be present in the form of a solvate, for example a hydrate. The solvate is also within the scope of the present invention.

The compound (1) of the present invention can be produced, for example, by the method shown below.

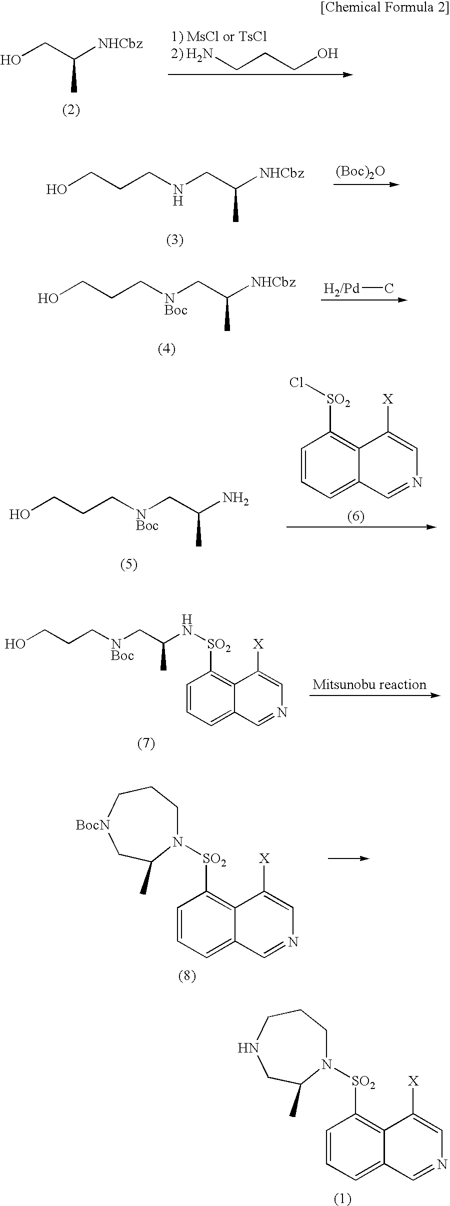

The compound (2), which is a starting material for the compound of the present invention is commercially available from Watanabe Chemical Industries, Ltd. or the like.

The reaction for obtaining the compound (3) from the compound (2) is carried out by converting the hydroxyl group of the compound (2) into methanesulfonyloxy group, tosyloxy group, or the like by a known method, and then allowing 3-amino-1-propanol to react with them in an appropriate solvent.

The methanesulfonylation or tosylation is carried out by allowing the compound (2) to react with methanesulfonyl chloride or the like in the presence of a tertiary amine, such as triethylamine. Examples of the reaction solvent used for the subsequent reaction with 3-amino-1-propanol include halogenated hydrocarbons, such as dichloromethane and chloroform; ethers, such as tetrahydrofuran (THF) and diethyl ether; N,N-dimethylformamide (DMF), acetonitrile, and the like. The reaction is carried out at 0° C. to around the boiling point of the solvent for 1 to 48 hours, preferably at 40° C. to 100° C. for 2 to 12 hours.

The amount of 3-amino-1-propanol used is 1 to 10 equivalents, preferably 4 to 6 equivalents, relative to the compound (2).

The amine of the resulting compound (3) is protected with a protecting group, such as a tert-butoxycarbonyl group, a formyl group, or a benzoyl group, to give the compound (4). The benzyloxycarbonyl group of the compound (4) is eliminated by hydrogenation in the presence of a metal catalyst, such as palladium, to give the compound (5). As the protecting group, the tert-butoxycarbonyl group is preferable.

The amino group-protecting reaction is carried out by allowing the compound (3) to react with the tert-butoxycarbonyl group or the like in the presence of a tertiary amine, such as triethylamine. The elimination reaction of the protecting group is carried out by adding hydrogen in an alcohol solvent in the presence of palladium-carbon.

The reaction of the primary amine (5) and the compound (6) is carried out in an appropriate solvent, preferably, in the presence of a necessary amount of a base; in which X is halogen, such as fluoro, chloro, bromo and iodo, especially bromo. Examples of the base include inorganic bases, such as potassium carbonate, sodium carbonate, and cesium carbonate; and organic bases, such as triethylamine, diisopropylethylamine, and triethylenediamine. The reaction solvent is the same as that used for obtaining the compound (3). The reaction is carried out at a temperature in the range of 0° C. to 80° C. for 0.5 to 24 hours, preferably at a temperature in the range of 10° C. to 50° C. for 1 to 8 hours.

The amount of use of the primary amine (5) is preferably 1 to 3 equivalents relative to the compound (6).

Note that the compound (6) can be synthesized by the method described in Japanese Unexamined Patent Application Publication No. 2-67274 or by a similar method.

The resulting compound (7) is subjected to ring closure, for example, by the Mitsunobu reaction using triphenylphosphine and an azodicarboxylate ester or the like to give the compound (8).

The deprotection reaction of the compound (8) is carried out by a known method suitable for the protecting group, for example, by acid treatment, alkaline treatment, or catalytic reduction. For example, when protected with the tert-butoxycarbonyl group, the compound (1) of the present invention can be obtained by treating the compound (8) with an ethyl acetate solution of hydrogen chloride, or the like.

The compound (1) of the present invention can be obtained by the method described above. Furthermore, if necessary, the compound (1) can be purified using a known purification technique, such as recrystallization or column chromatography. Furthermore, the compound (1) can be converted into the desired salt or solvate using a common technique.

As shown in Experimental Example 1 below, the compound (1) of the present invention thus obtained, its acid-added salt, or their solvates has a potent Rho-kinase inhibitory action and is useful as a therapeutic agent for treating diseases such as hypertension, pulmonary hypertension, cerebral vasospasm, angina pectoris, cardiac failure, arteriosclerosis, glaucoma, dysuria, asthma, and erectile failure.

The drug of the present invention contains, as an active ingredient, the compound (1) of the present invention, its acid thereof, or their solvates. The form of administration is not particularly limited and can be appropriately selected depending on the therapeutic application intended. For example, the drug may be administered in any form, for example, as an oral preparation, an injection, a suppository, an ointment, an inhalant, eye drops, nasal drops, or an adhesive preparation. A composition suitable for use in any of these administration forms can be prepared by blending a pharmaceutically acceptable carrier using any preparation method known to a person skilled in the art. It should be noted that the drugs may contain the compounds represented by formula (1) solely or in combination.

When an oral solid preparation is formulated, an excipient, and if necessary, a binder, a disintegrator, a lubricant, a colorant, a taste corrigent, a smell corrigent, and the like may be added to the compound (1) of the present invention, and the resulting composition can be formulated into tablets, coated tablets, granules, powders, capsules, etc. in the usual manner. As such additives, those which are used in the pharmaceutical field may be used. Examples include excipients, such as lactose, sucrose, sodium chloride, glucose, starch, calcium carbonate, kaolin, microcrystalline cellulose, and silicic acid; binders, such as water, ethanol, propanol, simple syrup, a glucose solution, a starch solution, a gelatin solution, carboxymethyl cellulose, hydroxypropyl cellulose, hydroxypropyl starch, methyl cellulose, ethyl cellulose, shellac, calcium phosphate, and polyvinyl pyrrolidone; disintegrators, such as dry starch, sodium alginate, powdered agar, sodium hydrogencarbonate, calcium carbonate, sodium lauryl sulfate, monoglyceryl stearate, and lactose; lubricants, such as purified talc, stearates, borax, and polyethylene glycol; and taste corrigents, such as sucrose, orange peel, citric acid, and tartaric acid.

When an oral liquid preparation is formulated, a taste corrigent, a buffer, a stabilizer, a smell corrigent, and the like may be added to the compound (1) of the present invention, and the resulting composition can be formulated into internal liquid preparations, syrup preparations, elixirs, etc. in a usual manner. In this case, as the taste corrigent, those mentioned above may be used. As the buffer, sodium citrate and the like may be used. As the stabilizer, tragacanth, gum arabic, gelatin, and the like may be used.

When an injection is formulated, a pH adjustor, a buffer, a stabilizer, an isotonizing agent, a local anesthetic, and the like may be added to the compound (1) of the present invention, and the resultant composition can be formulated into subcutaneous, intramuscular, and intravenous injections in a usual manner. In this case, examples of the pH adjustor and the buffer include sodium citrate, sodium acetate, and sodium phosphate. Examples of the stabilizer include sodium pyrosulfite, EDTA, thioglycolic acid, and thiolactic acid. Examples of the local anesthetic include procaine hydrochloride and lidocaine hydrochloride. Examples of the isotonizing agent include sodium chloride and glucose.

When a suppository is formulated, a pharmaceutical carrier known in the art, such as polyethylene glycol, lanoline, cacao butter, or fatty acid triglyceride, and if necessary, a surfactant, such as Tween (registered trademark), may be added to the compound (1) of the present invention, and the resultant composition can be formulated into suppositories in a usual manner.

When an ointment is formulated, a base material, a stabilizer, a wetting agent, a preservative, and the like, which are commonly used, may be blended with the compound (1) of the present invention if necessary, and the resulting composition may be mixed and formulated into ointments in a usual manner. Examples of the base material include liquid paraffin, white vaseline, bleached beeswax, octyldodecyl alcohol, and paraffin. Examples of the preservative include methyl p-hydroxybenzoate, ethyl p-hydroxybenzoate, and propyl p-hydroxybenzoate.

In addition to the above, inhalants, eye drops, and nasal drops may also be formulated in a usual manner.

While the dose of the drug of the present invention varies depending on the age, body weight, symptom, administration form, number of doses, and the like, usually, the drug is preferably administered orally or parenterally in one time or in several portions in a dose of 1 to 1,000 mg per day for an adult.

### EXAMPLES

The present invention will be described in detail below with reference to the examples. It is to be understood that the present invention is not limited thereto.

### Production Example 1

Synthesis of 2-(S)-2-(benzyloxycarbonyl)amino-N-(3-hydroxypropyl)propylamine (Compound 3):

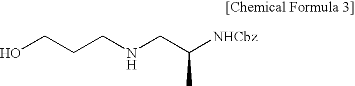

2-(S)-2-(Benzyloxycarbonyl)amino-1-propanol (4.96 g) and triethylamine (5.0 mL) were dissolved in chloroform (50 mL), and then methane sulfonyl chloride (2.7 mL) was added dropwise in an ice bath. The temperature of the reaction mixture was raised to room temperature, and the mixture was stirred for 30 minutes. Water was added to separate the organic layer. The aqueous layer was further extracted with chloroform, while the organic layers were combined, washed with saturated brine, and dried over anhydrous magnesium sulfate, followed by vacuum concentration. The residue was dissolved in THF (50 mL), and 3-amino-1-propanol (8.90 g) was added to the resulting solution. Refluxing was performed overnight. The mixture was subjected to vacuum concentration, and water and chloroform were added to separate the organic layer. The aqueous layer was further extracted with chloroform, while the organic layers were combined, washed with saturated brine and dried over anhydrous magnesium sulfate, followed by vacuum concentration. The residue was purified by silica gel column chromatography (developing solvent: chloroform/methanol=9/1→chloroform/ammonia-saturated methanol=9/1) to give the aimed compound as a colorless oily substance.

Yield: 3.76 g (60%)

### Production Example 2

Synthesis of 2-(S)-2-(benzyloxycarbonyl)amino-N-(tert-butoxycarbonyl)-N-(3-hydroxypropyl)propylamine (Compound 4):

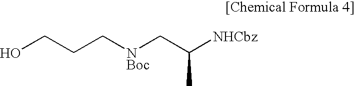

2-(S)-2-(Benzyloxycarbonyl)amino-N-(3-hydroxypropyl)propylamine (3.76 g) and triethylamine (2.4 mL) were dissolved in chloroform (20 mL), and di-tert-butyl dicarbonate (3.70 g) was added, followed by stirring at room temperature. After completion of the reaction, the mixture was subjected to vacuum concentration, and ethyl acetate and water were added to the residue to separate the organic layer. The aqueous layer was further extracted with ethyl acetate, while the organic layers were combined, washed with saturated brine and dried over anhydrous sodium sulfate, followed by vacuum concentration. The residue was purified by silica gel column chromatography (developing solvent: n-hexane/ethyl acetate=10/1→n-hexane/ethyl acetate=1/1) to give the target compound as a colorless oily substance.

Yield: 4.38 g (85%)

### Production Example 3

Synthesis of 2-(S)-2-amino-N-(tert-butoxycarbonyl)-N-(3-hydroxypropyl)propylamine (Compound 5):

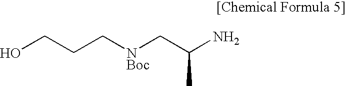

2-(S)-2-(Benzyloxycarbonyl)amino-N-(tert-butoxycarbonyl)-N-(3-hydroxypropyl)propylamine (4.38 g) was dissolved in methanol (20 mL), and catalytic reduction was carried out in the presence of 10% palladium-activated carbon (440 mg) under hydrogen flow. After completion of the reaction, the catalyst was removed and the filtrate was subjected to vacuum concentration to give the aimed compound as a colorless oily substance.

Yield: 2.77 g (100%)

### Production Example 4

Synthesis of 2-(S)-2-(4-bromoisoquinoline-5-sulfonylamino)-N-(tert-butoxycarbonyl)-N-(3-hydroxypropyl)propylamine (Compound 7, in which X is Br):

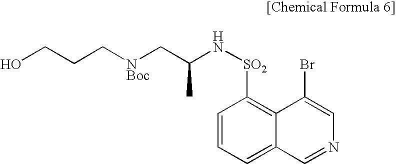

2-(S)-2-Amino-N-(tert-butoxycarbonyl)-N-(3-hydroxypropyl)propylamine (534 mg) and triethylamine (390 μL) were dissolved in chloroform (5 mL), and 4-bromoisoquinoline-5-sulfonyl chloride (593 mg) was added, followed by stirring at room temperature. After completion of the reaction, water was added to the mixture to separate the organic layer. The aqueous layer was further extracted with chloroform, while the organic layers were combined, washed with saturated brine and dried over anhydrous magnesium sulfate. After vacuum concentration was carried out, the residue was purified by silica gel column chromatography (developing solvent: ethyl acetate→ethyl acetate/acetone=4/1) to give the aimed compound as a colorless oily substance.

Yield: 454 mg (47%)

### Production Example 5

Synthesis of 2-(S)-1-(4-bromoisoquinoline-5-sulfonyl)-4-N-(tert-butoxycarbonyl)-2-methylhomopiperazine (Compound 8, in which X is Br):

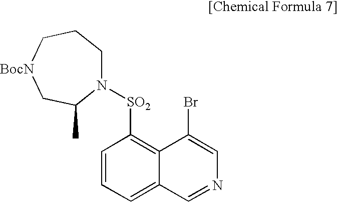

2-(S)-2(4-Bromoisoquinoline-5-sulfonylamino)-N-(tert-butoxycarbonyl)-N-(3-hydroxypropyl)propylamine (454 mg) and triphenylphosphine (356 mg) were dissolved in anhydrous THF. In an argon atmosphere, a 40% toluene solution (590 mg) of diethyl azodicarbonate was added dropwise to the solution, which was stirred overnight at room temperature. After the mixture was subjected to vacuum concentration, chloroform and water were added to the residue to separate the organic layer. The aqueous layer was further extracted with chloroform, while the organic layers were combined, washed with saturated brine and dried over anhydrous magnesium sulfate, followed by vacuum concentration. The residue was purified by silica gel column chromatography (developing solvent: n-hexane/ethyl acetate=1/1→ethyl acetate). Since it was difficult to separate the aimed compound from the side product, the resulting product was used in the subsequent step without further purification.

Yield: 462 mg (including impurities)

### Example 1

Synthesis of dihydrochloride of 2-(S)-1-(4-haloisoquinoline-5-sulfonyl)-2-methylhomopiperazine (Compound 1):
2-(S)-1-(4-bromoisoquinoline-5-sulfonyl)-2-methylhomopiperazine (X is Br);

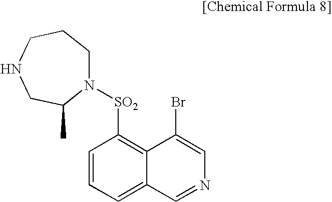

The crude 2-(S)-4-(tert-butoxycarbonyl)-1-(4-bromoisoquinoline-5-sulfonyl)-2-methylhomopiperazine (164 mg) obtained by cyclization of 104 mg of the alcohol in the previous step was dissolved in methanol (1 mL), and an ethyl acetate solution (2 mL) of 4 M hydrochloric acid was added. After completion of the reaction, precipitated crystals were collected and washed with ethyl acetate on a funnel to give the aimed compound.

Yield: 71 mg (74%, 2 steps) ¹H-NMR (270 MHz, DMSO-d₆, 100° C.) δ: 1.25 (d, 3H, J=7.0 Hz), 2.02-2.15 (m, 2H), 3.12-3.44 (m, 4H), 3.62-3.67 (m, 2H), 4.33-4.43 (m, 1H), 7.86 (t, 1H, J=7.8 Hz), 8.33 (dd, 1H, J=7.6 Hz, 1.4 Hz), 8.45 (dd, 1H, J=7.8 Hz, 1.1 Hz), 8.92 (s, 1H), 9.38 (s, 1H).

According to the same manner as the synthesis of 2-(S)-1-(4-bromoisoquinoline-5-sulfonyl)-2-methylhomopiperazine (Compound 1: X is Br) described above, other 2-(S)-1-(4-haloisoquinoline-5-sulfonyl)-2-methylhomopiperazines (Compound 1: X is F or Cl) were obtained.

### Experimental Example 1

### Measurement of Kinase Inhibitory Activity

A Rho-kinase assay was performed according to the method described in Patent Document 1, and the 50% inhibitory concentration (hereinafter referred to as the “IC₅₀ value”) for Rho kinase was calculated.

The IC₅₀ value for the Rho kinase of the dihydrochloride of the compound (1) in Example is shown below together with the IC₅₀ values of Reference Compound A (fasudil hydrochloride: hexahydro-1-(5-isoquinolinesulfonyl)-1H-1,4-diazepine hydrochloride) and Reference Compound B ((S)-(+)-hexahydro-2-methyl-1-(4-methyl-5-isoquinolinesulfonyl)-1H-1,4-diazepine hydrochloride).

TABLE 1

| Compound | | IC₅₀ (μM) |
|---|---|---|
| Dihydrochloride of Compound (1) | X = Br | 0.0058 |
| | X = Cl | 0.0258 |
| | X = F | 0.085 |

| | |
|---|---|
| Reference Compound A | 0.158 |
| Reference Compound B | 0.012 |

Specific preparation examples will be shown below.

### Preparation Example 1 (Capsule)

| | |
|---|---|
| Compound of Example 1 | 30 mg |
| Microcrystalline cellulose | 30 mg |
| Lactose | 57 mg |
| Magnesium stearate | 3 mg |
| Total | 120 mg |

The above ingredients were mixed in a usual manner, and the resulting mixture was filled into a gelatin capsule, and a capsule was thus obtained.

### Preparation Example 2 (Tablet)

| | |
|---|---|
| Compound of Example 1 | 30 mg |
| Starch | 44 mg |
| Starch (for paste) | 5.6 mg |
| Magnesium stearate | 0.4 mg |
| Carboxymethyl cellulose calcium | 20 mg |
| Total | 100 mg |

### Preparation Example 3 (Injection)

The compound (100 mg) of Example 1 and sodium chloride (900 mg) were dissolved in about 80 mL of distilled water for injection, and distilled water for injection was added to make the total volume 100 mL. The resulting solution was aseptically filtered, and then divided into 10 ampoules. The ampoules were sealed to obtain aseptic injections.

1. A method for treating a disease caused by the activation of Rho kinase, comprising administering a 4-haloisoquinoline derivative represented by formula (1), an acid-added salt thereof, or a solvate of any of the foregoing: 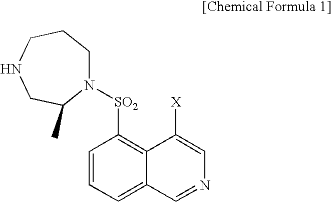
wherein X is halogen.

2. The method of claim 1, wherein X is Br.

3. The method of claim 1, wherein the disease is selected from the group consisting of hypertension, pulmonary hypertension, cerebral vasospasm, angina pectoris, cardiac failure, arteriosclerosis, glaucoma, dysuria, asthma, erectile failure.

4. A method for activating a Rho kinase, comprising administering a 4-haloisoquinoline derivative represented by formula (1), an acid-added salt thereof, a solvate of any of the foregoing: 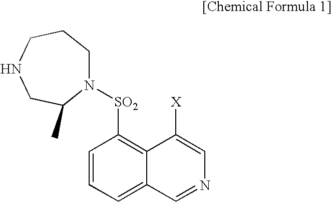
wherein X is halogen.

5. The method of claim 4, wherein X is Br.
